(19) United States
(12) Patent Application Publication
(10) Pub. No.: US 2025/0377912 A1
(43) Pub. Date: Dec. 11, 2025

(54) Selective Determination of Presentation Modes for Observation Data Elements

(71) Applicant: Apple Inc., Cupertino, CA (US)

(72) Inventor: Zachary J. Waterson, San Francisco, CA (US)

(73) Assignee: Apple Inc., Cupertino, CA (US)

(21) Appl. No.: 19/098,168

(22) Filed: Apr. 2, 2025

Related U.S. Application Data
(60) Provisional application No. 63/657,654, filed on Jun. 7, 2024.

(51) Int. Cl. G06F 9/451; G06F 3/0485; G06T 13/80

(52) U.S. Cl. CPC G06F 9/451; G06T 13/80; G06F 3/0485

(57) ABSTRACT

Techniques described herein relate to selective determination of presentation modes for observation data elements. An example process may include receiving a new observation data element of a first data element data category. The process may also include adding the new observation data element to a data model stored on the user device. The process may also include generating bundled observation data based on the new observation data element. The process may also include providing the bundled observation data to a view layer of the application. The process may also include evaluating the bundled observation data in accordance with at least one evaluation criteria of a plurality of evaluation criteria to determine a presentation mode relating to presentation of information associated with the bundled observation data. The process may also include. in response to receiving a view input request, implementing the presentation mode at a user interface of the user device.

### CROSS-REFERENCES TO RELATED APPLICATIONS

This application claims the benefit of U.S. Provisional Application No. 63/657,654, filed on Jun. 7, 2024, which is incorporated by reference.

### BACKGROUND

Electronic devices, especially portable electronic user devices, are quickly becoming ubiquitous in every modern society. Such devices can be used to collect and store personal information, such as health data, about a user. Certain health data may be updated periodically or in response to a trigger (e.g., when new data is received). With health data coming from different sources, combined with the potential for large amounts of data, it can present challenges for maintaining the most current data and for informing the user when data changes.

### BRIEF SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a computer-implemented method. The computer-implemented method includes receiving a new observation data element of a first data element data category. The computer-implemented method also includes adding the new observation data element to a data model stored on the user device. The computer-implemented method also includes generating bundled observation data based on the new observation data element, the bundled observation data representing a new state for the new observation data element. The computer-implemented method also includes providing the bundled observation data to a view layer of the application. The computer-implemented method also includes evaluating the bundled observation data in accordance with at least one evaluation criteria of a plurality of evaluation criteria to determine a presentation mode relating to presentation of information associated with the bundled observation data. The computer-implemented method also includes, in response to receiving a view input request, implementing the presentation mode at a user interface of the user device. Other embodiments of this aspect include corresponding computer systems, apparatus, and computer programs recorded on one or more computer storage devices, each configured to perform the actions of the methods.

### BRIEF DESCRIPTION OF THE DRAWINGS

FIG. 1 illustrates a block diagram and a flowchart showing an example process for selective determination of presentation modes for observation data elements, according to at least one example.

FIG. 2 illustrates a diagram showing a data model layer portion of an example process for selective determination of presentation modes for observation data elements, according to at least one example.

FIG. 3 illustrates a block diagram and a flowchart showing a view layer portion of the example process from FIG. 2, according to at least one example.

FIG. 4 illustrates an example user interface view of a home screen of a portable user device, according to at least one example.

FIG. 5 illustrates an example user interface view of a health application including updatable user interface elements relating to selective determination of presentation modes for observation data elements, according to at least one example.

FIG. 6 illustrates a zoomed-in view of an example updatable user interface element from FIG. 5, according to at least one example.

FIGS. 7A-7C illustrate various states of a set of updatable user interface elements from FIG. 5, according to various examples.

FIG. 8 illustrates a flow chart showing an example process for selective determination of presentation modes for observation data elements, according to at least one example.

FIG. 9A illustrates a flow chart showing an example process for selective determination of presentation modes for observation data elements, according to at least one example.

FIG. 9B illustrates a flow chart showing an example process for selective determination of presentation modes for observation data elements, according to at least one example.

FIG. 9C illustrates an example device configured to implement techniques described herein, according to at least one example.

FIG. 9D illustrates an example system configured to implement techniques described herein, according to at least one example.

FIG. 9E illustrates a flow chart corresponding to FIG. 9A showing an example process for selective determination of presentation modes for observation data elements, according to at least one example.

FIG. 9F illustrates a flow chart corresponding to FIG. 9B showing an example process for selective determination of presentation modes for observation data elements, according to at least one example.

FIG. 10 illustrates an example architecture or environment configured to implement techniques described herein, according to at least one example.

### DETAILED DESCRIPTION

In the following description, various examples will be described. For purposes of explanation, specific configurations and details are set forth in order to provide a thorough understanding of the examples. However, it will also be apparent to one skilled in the art that the examples may be practiced without the specific details. Furthermore, well-known features may be omitted or simplified in order not to obscure the example being described.

Examples of the present disclosure are directed to, among other things, methods, systems, devices, and computer-readable media to intelligently maintain a state of health data for a user and use the state and other data characteristics to selectively determine a presentation mode for presenting updates to the health data. The health data may include health and fitness-related information that is stored and processed on a user device, in accordance with a predefined framework (e.g., HealthKit framework). The health data may be processed by one or more user devices and synced across the devices using a cloud platform. In some examples, a user device may include a dedicated application that processes health data, stores health data, and includes at least some functionality relating to presenting the health data at a user interface of the user device.

The health data may represent various health-related metrics. The value of these metrics may change over time. Some may change more frequently than others. For example, step data, heart rate data, and similar data types may be more or less streamed in real-time to the user device or even collected by sensors of the user device. Other data such as weight, blood-related metrics, and the like may be updated when the user sees a physician or takes a body measurement, which may occur less frequently. The application described herein may be used to track when the data changes and present such changes to the user. To avoid providing a notification to the user every time any data is updated, which can lead to notification fatigue, the techniques described herein may track the state of the data, evaluate what type of change has occurred, and then determine an appropriate presentation mode for presenting the update.

The presentation mode, which can include a graphical animation, can function as a proxy for sending a notification or otherwise alerting the user. An example presentation mode may include visually depicting how a step count has incremented up since last viewed by the user. Various triggers may be defined to determine when to execute a presentation mode. For example, when the user first opens the application, certain values may be animated as changing. If data comes in while the application is open, the application may refrain from animating increases. This might be helpful to conserve battery life and to avoid distracting the user. However, if the user performs an action that causes the application content to refresh (e.g., pulling down on the user interface, closing then reopening the application, selecting a “refresh” user interface element, or the like), the presentation mode including an animation may be implemented to show the changes.

Turning now to a particular example, in this example there is provided a system including a wearable user device such as a smartwatch and a portable user device such as a tablet. When worn, the wearable user device may collect health data from a wearer (e.g., heartrate, step count, calories burned, etc.). These health data (or some processed version of the health data) may be shared with the tablet. For example, the health data may be shared via any suitable network connection, which may include a cloud synching service. Once the health data is received by the tablet, the tablet may identify a state of the health data and compare it to a state of old health data of the same type already stored in a health database on the tablet. Part of doing so, may include the tablet combining the health data with the old health data into a bundle, which may represent at least the state of the health data. The tablet may evaluate this bundle using various evaluation criteria to determine whether to update a user interface at the tablet with the new health data. In addition, the tablet may evaluate the bundle to determine a particular presentation mode for presenting the new health data. The presentation modes may include various graphical animations to indicate changes in the health data to the user. For example, if the health data were updated heart rate values and it was determined that the user's heartrate had increased as compared to the old health data, the selected presentation mode may include a presentation mode including a graphical animation that increments up the value of the heartrate displayed (e.g., sometimes referred to herein as a “ticker up animation”). This graphical animation may be configured to draw the user's attention to the updated value.

The examples described herein address a number of technical problems and provide for a number of technical improvements. In some examples, these improvements additionally improve the functioning of various components of a system in which the techniques are implemented. The techniques described herein provide for customized presentation of health data, while also reducing on-device storage requirements and processing requirements for generating user interfaces. This is because use of the selective determination of presentation modes for observation data elements (e.g., health data) enables the system to quickly disregard data that is not new. Moreover, by using evaluation criteria that dynamically pick presentation modes, including a mode that does not include graphical animations, the system avoids wasting computing resources updating graphical user interfaces when no new health data is present.

The techniques described herein also provide for more computationally efficient and user efficient navigation of computer applications and operating systems. For example, the presentation modes, including the graphical animations, are presented at an appropriate time and in a way that quickly captures the user's attention to see the update, but does not require the user to perform additional functions to see the updates. For example, when a user opens an application that stores the health data, a user interface including the health data can be automatically updated with a graphical animation that highlights the updates. This prevents the user from having to click a refresh button or drill down into the data to see the updates. The user interface elements in which the graphical animations take place may have been previously selected by the user and “pinned” at a prominent location in the application. Thus, rather than the user having to dive deeper into the application (e.g., multiple selections, page throughs, click throughs, tabs, etc.), the information that is relevant and important to the user is prominently displayed and, when the conditions are right, dynamically updated.

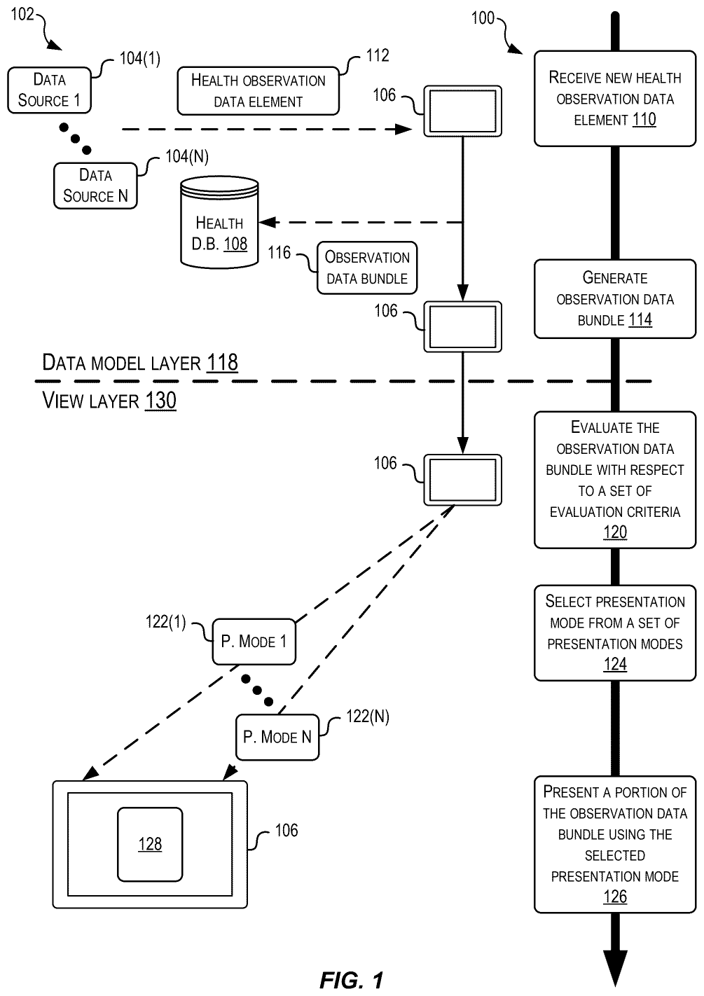

Turning now to the figures, FIG. 1 illustrates a block diagram 102 and a flowchart showing a process 100 for selective determination of presentation modes for observation data elements, according to at least one example. The diagram 102 depicts a computer system that includes data sources 104(1)-104(N), a user device 106, and a health database 108. These system elements may be distributed between other devices and/or may be implemented in one device. For example, a wearable device may function as a data source (e.g., includes sensors that collect health data), include a database for storing health data (e.g., the health database 108), and may perform the process 100 relating to the presentation of health data.

The data sources 104(1)-104(N) (collectively referred to as “data source 104” or “data sources 104”) may be any suitable system, device, sensor, or the like capable sharing health data such as a health observation data element 112 with the user device 106. As an example, a data source 104 may be a wearable device that collects health signals using sensors and processes the health signals to derive health data. As an additional example, a data source 104 may be another user device 106 that captures or otherwise maintains health data. In some examples, the user device 106 and another user device 106 may be associated with the same user profile (e.g., account) or may be associated with different user profiles but otherwise associated (e.g., spouses with separate accounts and separate devices but who have agreed to share health data with each other). As an additional example, a data source 104 may be a health provider system, service, or the like. For example, the data source 104 may be a gateway of an electronic medical record (EMR) system, and the user device 106 may be used to access health data from the gateway. In some examples, the user device 106 may establish a persistent connection with the gateway such that when health data is updated by the EMR system (e.g., chart data, sample data, test data, blood work, etc.), that data is shared over the persistent connection with the user device 106. The data sources 104 may be connected to the user device 106 via any suitable network including, for example, the Internet, Wi-Fi, cellular, and the like.

The user device 106, which may be any suitable device such as a smartphone, tablet, smartwatch, wearable device, laptop computer, or desktop computer, may be configured to interact with the data sources 104. For example, the user device 106 may include one or more network radios to enable network connections with the data sources 104. In some examples, the user device 106 may include one or more applications, which may include custom-built algorithms and other logic, to enable performance of at least some of the techniques described herein. The user device 106 may also include storage media for storing computer-executable instructions (e.g., that make up the application) and other data such as described herein. The user device 106 may be operated by a user. The health database 108 may be resident on the user device 106 and may be used to store health data relating to the techniques described herein, including at least aspects of health observation data elements.

In some examples, the data sources 104, the health database 108, and user device 106 may be implemented by a single entity (e.g., part of a broader hardware and software ecosystem of interconnected hardware, software, and services provided by a service provider). In some examples, the data sources 104, the health database 108, and the user device 106 may be implemented by different service providers. For example, the data sources 104 may be manufactured by a first entity and may be designed to communicate with the user device 106, which may be manufactured a second entity.

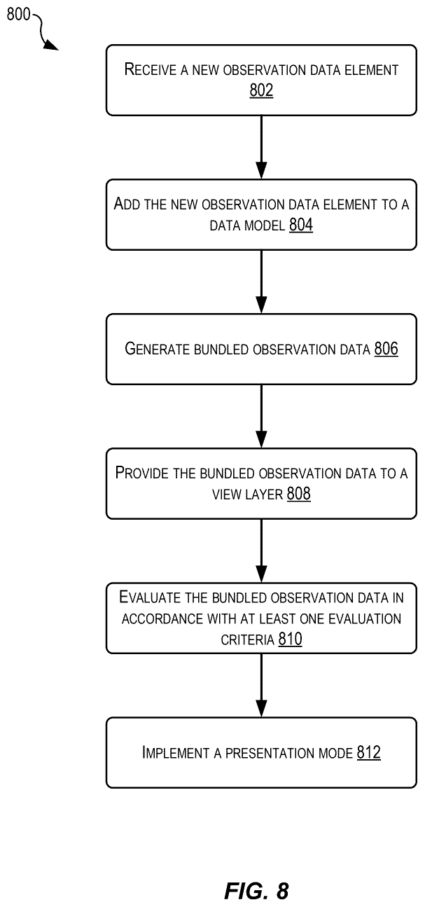

FIGS. 1 and 8 illustrate example flow diagrams showing processes 100 and 800, according to at least a few examples. These processes, and any other processes described herein, are illustrated as logical flow diagrams, each operation of which represents a sequence of operations that can be implemented in hardware, computer instructions, or a combination thereof. In the context of computer instructions, the operations may represent computer-executable instructions stored on one or more non-transitory computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Additionally, some, any, or all of the processes described herein may be performed under the control of one or more computer systems configured with specific executable instructions and may be implemented as code (e.g., executable instructions, one or more computer programs, or one or more applications) executing collectively on one or more processors, by hardware, or combinations thereof. As noted above, the code may be stored on a non-transitory computer-readable storage medium, for example, in the form of a computer program including a plurality of instructions executable by one or more processors.

The process 100 begins at block 110 by the user device 106 receiving a new health observation data element 112 from the one or more data sources 104. In some examples, the health observation data element 112 may include a standardized set of data types and/or data categories defined by a health framework.

In some examples, the health observation data element 112 may include various types of health data which may include, for example, activity data (e.g., step counts, distances walked, flights of stairs climbed, energy expended, etc.), medical records (e.g., allergies, medications, medical conditions, immunizations, lab results, etc.), workout data (e.g., details about workouts or exercises, including length type of activity and intensity), biometric data (e.g., heart rate, blood pressure, respiratory rate, body temperature, blood glucose levels, etc.), nutrition data (e.g., calorie intake, nutrient consumption, nutrient levels, etc.), sleep data (e.g., sleep duration, sleep quality, sleep patterns, etc.), sound data (e.g., values and exposure to sound), stability data (e.g., values surrounding how stabile a user is), and any other suitable health-related data. In some examples, the new health observation data element 112 may include structured data entries from a user's health record stored by an EMR system. The health observation data element 112 may include state information in the form of metadata, header information, timestamps, or the like that is useable to compare the state of the health observation data element 112 with other data.

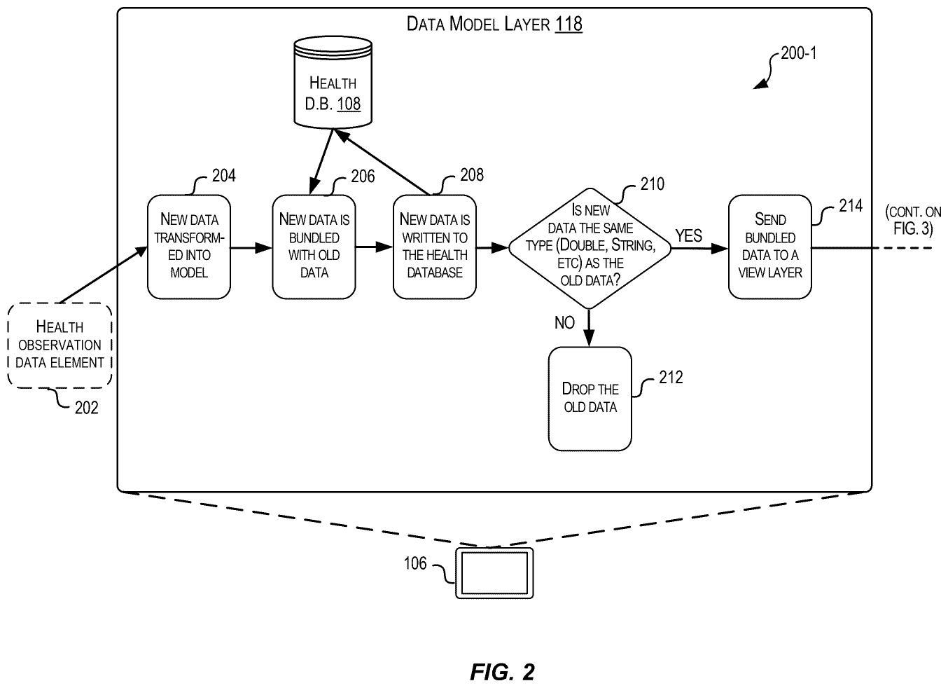

At block 114, the process 100 includes the user device 106 generating an observation data bundle 116 using the new health observation data element. Generating the observation data bundle 116 may include combining the new health observation data element 112 with an older version of the health observation data element 112 stored in the health database 108 and available to the user device 106. The two data elements 112 may be combined to define the observation data bundle 116, which may represent the old state of the data element and the new state of the data element. As described in more detail in FIG. 2, in some examples, blocks 110 and 114 may be performed at a data model layer 118 of an application or operating system of the user device 106.

At block 120, the process 100 includes the user device 106 evaluating the observation data bundle with respect to a set of evaluation criteria. This may include evaluating the new state and the old state, evaluating differences and similarities between the old and new data in the observation data bundle 116. The purpose of block 120 is to identify potential presentation modes 122 and to eliminate incompatible presentation modes 122, so that at block 124, the user device 106 can select a presentation mode 122 from a set of presentation modes 122(1)-122(N) (collectively referred to as “presentation mode 122” or “presentation modes 122”). The presentation modes 122 may correspond to a graphical animation technique, a decision to not present, a decision to not present with a graphical animation, and/or any suitable combination of the foregoing.

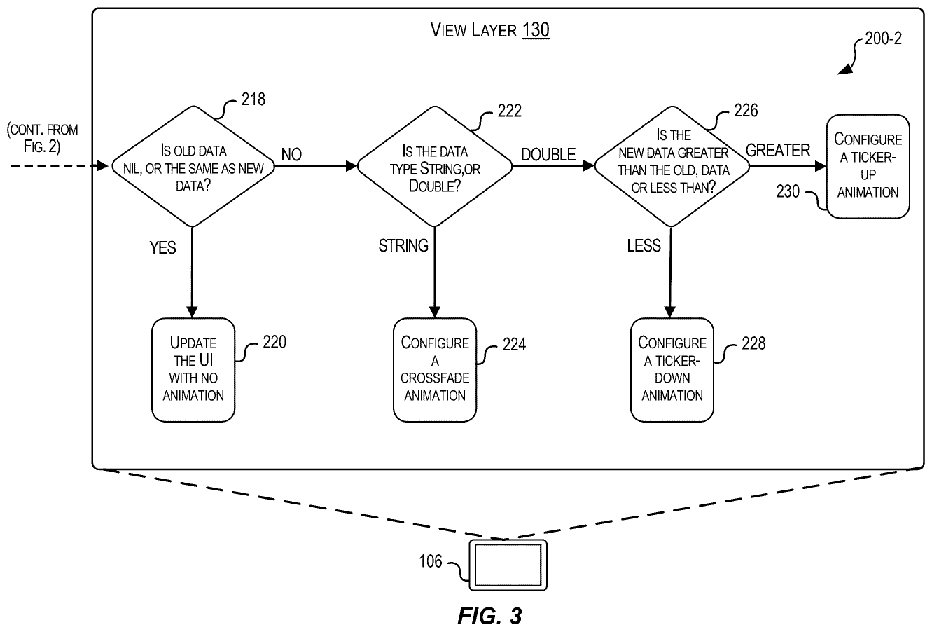

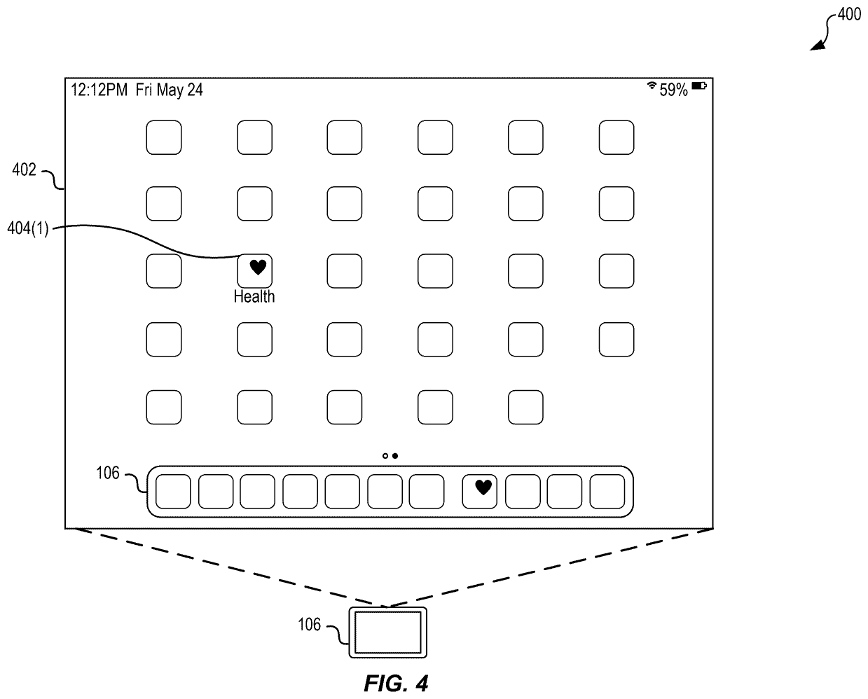

At block 126, the process 100 may include the user device presenting a portion of the observation data bundle using the selected presentation mode 122. This may include updating a graphical user interface 128 of the user device 106 to implement the presentation mode 122, as shown in more detail in FIGS. 4-7. As described in more detail in FIG. 3, in some examples, blocks 120, 124, and 126 may be performed at a view layer 130 of an application or operating system of the user device 106.

While not explicitly shown in FIG. 1, the process 100 may also include the user device 106 processing the health observation data element 112. This may include indexing, organizing, and otherwise adjusting the health observation data element 112 to improve the presentation of the health observation data element 112 at block 126.

FIG. 2 illustrates a diagram showing a data model layer portion of an example process 200 (e.g., process 200-1) for selective determination of presentation modes for observation data elements, according to at least one example. The process 200-1 may be implemented within the data model layer 118 of the user device 106. Like in FIG. 1, the process 200 may be begin, at 202, with a health observation data element being received at the data model layer 118 of the user device 106. At block 204, the process 200-1 may include the user device 106 transforming the new data (e.g., the health observation data element) into a data model on the user device 106. The data model may include a health data model used by the user device 106 for storing and organizing health data according to a set of predefined concepts, categories, and the like. In some examples, the data model may be one or more data models and may include a relational data model, a document-oriented data model, a hierarchical data model, a graph data model, and/or a time series data model. In some examples, the selection of the data model may depend on the type of the health observation data element.

The data model layer 118 may represent the underlying structure and organization of the data used by an application. In some examples, the data model layer 118 may include classes or structures that define the data types, properties, and relationships between different pieces of data. The data model layer 118 may be responsible for managing data persistence, retrieval, and manipulation. In some examples, the data model layer 118 may be implemented using Swift classes or structures that conform to various protocols, such as Codable for serialization/deserialization or CoreData for managing object graphs and persistent storage.

At block 206, the process 200-1 may include the user device 106 bundling the new data with the old data. This may include accessing the old data from the health database 108. The new data may include the health observation data element and the old data may include an old corresponding health observation data element. Bunding the data may correspond to generating a bundle or otherwise combining the data in a way that it can be compared at subsequent steps.

At block 208, the process 200-1 may include the user device 106 writing new data to the health database 108. This may include storing the new data to the health database 108.

At block 210, the process 200-1 may include the user device 106 determining whether the new data is the same data type as the old data. In this example, the data type may correspond to different types of data that can be manipulated by a program. Each data type defines that values that a variable can hold. Example data types may include, integer data types (e.g., int. long, short, byte, etc.), floating-point data types (e.g., float, double, etc.), character type data types (e.g., char, etc.), Boolean data types (e.g., bool), string data types (e.g., string, char[ ], etc.), and any other suitable data type.

If the data type of the new data is not the same as the old data, then the process 200-1 proceeds to block 212. At block 212, the process 200-1 may include the user device 106 dropping the old data. In other words, the old data may be disregarded. If the data type of the new data is the same as the old data, then the process 200-1 proceeds to block 214. At block 214, the process 200-1 may include the user device 106 sending bundled data to the view layer 130. The operations that occur at the view layer 130 will be described with respect to FIG. 3.

FIG. 3 illustrates a diagram showing a view layer portion of an example process 200 (e.g., the process 200-2) for selective determination of presentation modes for observation data elements, according to at least one example. The process 200-2 may be implemented within the view layer 130 by the user device 106. The process 200-2 corresponds to selecting a presentation option using a set of evaluation criteria, which may depend on various characteristics of the bundle, including, for example, data type characteristics of the old data and the new data.

In some examples, the view layer 130 may be responsible for presenting the user interface (UI) of the application and handling user interactions. The view layer 130 may include all visual elements such as buttons, labels, text fields, and views with which users interact. The view layer 130 may be primarily concerned with the presentation and layout of UI components based on the data provided by the data model layer 118. In some examples, the view layer 130 may be implemented using Interface Builder or programmatically using UIKit.

The process 200-2, which is a continuation from block 216 in FIG. 2 of process 200-1, may begin at block 218. At block 218, the process 200-2 may include the user device 106 evaluating a first set of evaluation criteria, which include determining whether the old data is nil or the same as the new data. This may include the user device comparing state values for the old data with state values for the new data. Having the view layer compare the values may provide for more dynamic control of when graphical animations are presented. For example, the user device 106 may use user input signals (e.g., received at a user interface) as an additional input to determine whether or not the data has changed.

For example, in cases where a user input signal indicates that a user has navigated to a “new” view, e.g., a user interface view that is different from the one that includes the health data, the old data will be nil, in which case the process 200-2 proceeds to block 220. At block 220, the process 200-2 may include the user device 106 updating the user interface with no animation. In other words, the selected presentation mode from block 218 would be no animation.

As an additional example, in cases where a user input signal indicates that a user has scrolled off screen then back on screen, the old data will equal the new data, in which case the process 200-2 proceeds to block 220. Similarly as described previously, at block 220, no animation is presented. This is because neither of these actions triggered new data and/or new data was not received. Or, if new data had been received, the logic at block 218 will prevent the animations from triggering too frequently, which could be distracting to users. For example, consider an example in which the incoming health data is heart rate data that is more or less streaming to the user device 106. In this example, new data is arriving in almost real-time and at a high frequency, but using the evaluation criteria described herein, the process 200-2 will decide when to animate and when to just update the user interface (e.g., change the value of the heart rate) without doing so using an animation.

If the answer at block 218 is NO, the process 200-2 may proceed to block 222. At block 222, the process 200-2 may include determine whether a data type of the bundle is a string type or a double type (e.g., or any other a floating-point data type). In some examples, block 222 may include evaluation criteria relating to different data types. Block 222 may be useful for the user device 106 to determine the presentation mode and thereby the best animation for the data type.

For example, if the data type of the health data is a string, the process 200-2 may proceed to block 224, at which, the presentation mode may include configuring a crossfade animation. A crossfade animation may be a visual effect that smoothly transitions from first visual information (e.g., a first string) to second visual information (e.g., a second string). The crossfade may enter the view using fading in and fading out, moving from a side, top, or bottom of the view, or in any other suitable manner. For example, the first visual information may gradually become transparent while the second element simultaneously becomes more opaque, creating a more or less seamless blend from one to the other. The crossfade animation may include defined initial states of opacity for the first and second information, a transition process that defines how the opacity changes, final states of opacity for the first and second information, and a duration for making the changes.

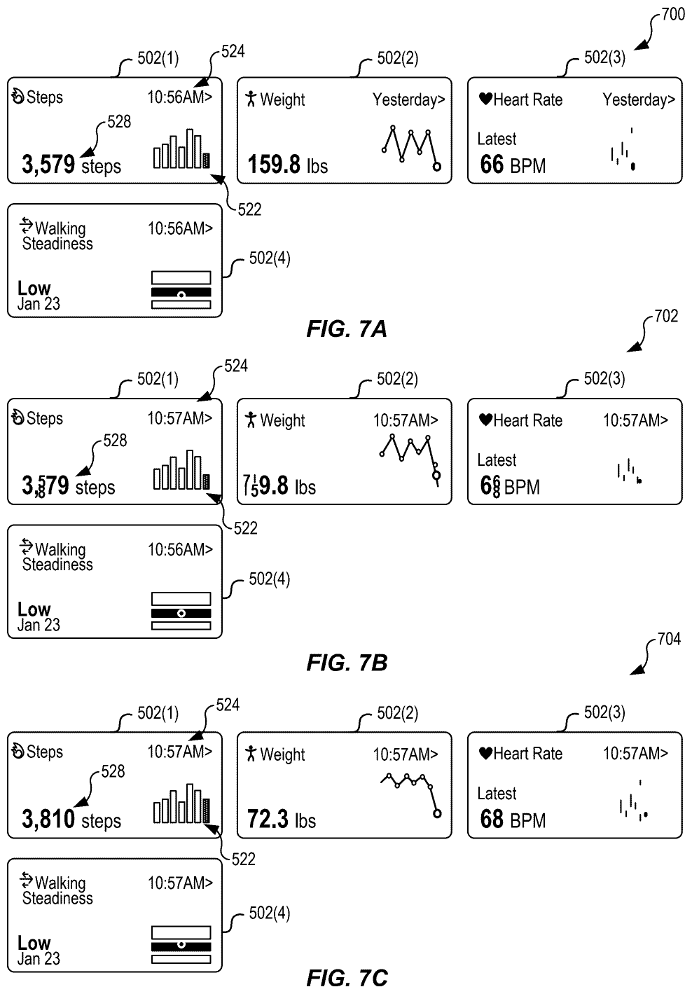

Returning to block 222, if the data type is a double or other floating-point data type, the process 200-2 may proceed to block 226 for evaluation of additional evaluation criteria. At block 226, the process 200-2 may include the user device 106 using the values of the old data and/or the new data (e.g., the floating-point data) to determine a presentation mode. For example, block 226 may include determining whether the new data (e.g., a value of the new data) is greater than the old data (e.g., a value of the old data) or less than the old data (e.g., a value of the old data). If the new data is less than the old data, the process 200-2 proceeds to block 228, which identifies a presentation mode including a ticker-down animation and includes the user device 106 configuring the ticker-down animation. The ticker-down animation, which is shown in more detail in FIG. 7, may include incrementing down the value of the old data to the value of the new data (e.g., “ticking” down the values). A ticker-down animation, which is similar to the ticker-up animation described with respect to block 230, may include a visual effect to display information by scrolling text or images vertically downward. It results in a “stream” like effect that is compact, non-intrusive, and attention grabbing.

If the new data is greater than the old data, the process 200-2 proceeds to block 230, which identifies a presentation mode including a ticker-up animation and includes the user device 106 configuring the ticker-up animation. The ticker-up animation may increment up the value of the old data to the value of the new data (e.g., “ticking” up the values).

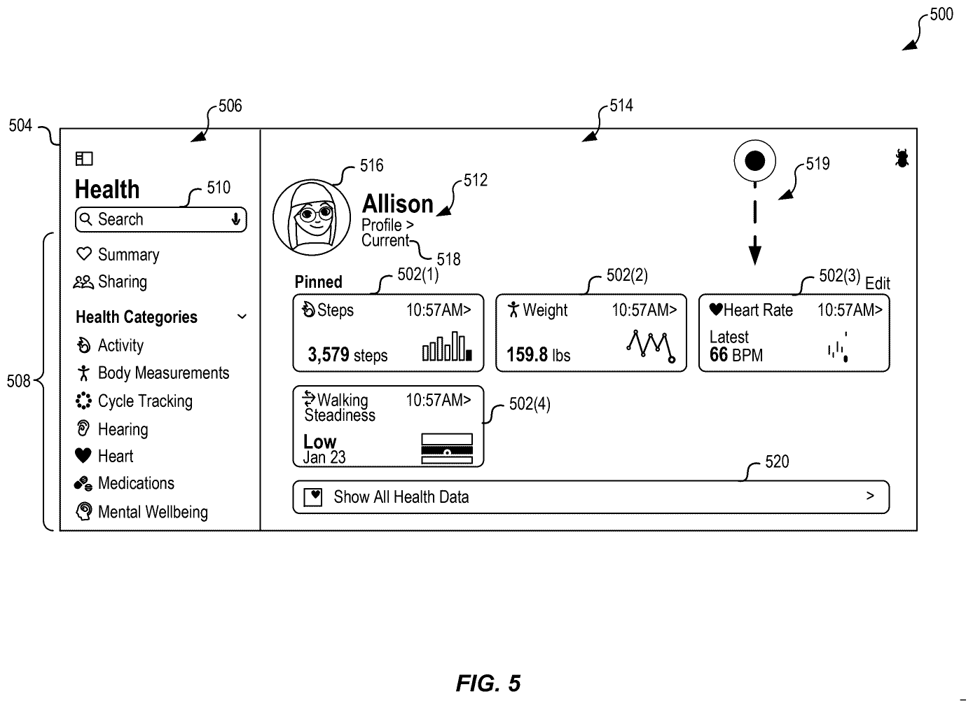

FIG. 4 illustrates an example user interface view 400 of a home screen 402 of the portable user device 106, according to at least one example. The home screen 402 may include a plurality of icons 404, which correspond to a plurality of applications. The icon 404(1) may correspond to a health application that implements at least some of the techniques described herein. In some examples, user selection of the icon 404(1) may cause the user device 106 to open the health application and present a landing page of the health application, which is depicted as a summary view in FIG. 5. In some examples, opening the health application may trigger any suitable action such as any block in process 200. In some examples, the operations at the data layer (e.g., process 200-1) may be performed in the background and operations in the view layer (e.g., process 200-2) may be performed in response to a user input signal (e.g., opening the health application by selecting the icon 404(1) or other suitable action as described herein). In some examples, separating the actions performed by the data layer and the view layer as it relates to presenting the health data may be beneficial for presenting the most relevant information in a way that captures the user's attention when needed but that is not overly obtrusive.

Turning now to FIG. 5, as introduced herein, FIG. 5 illustrates an example user interface view 500 of a health application including updatable user interface elements 502 relating to selective determination of presentation modes for observation data elements, according to at least one example. The user interface view 500 may include a summary view 504 be presented at the user device 106 in response to user selection of the icon 404(1) or in any other suitable manner (e.g., via a link from a different application, via a tray of recently opened applications, and the like).

The summary view 504 may include a category area 506 that includes a category list 508 and search bar 510. The category list 508 may include a list of selectable icons to navigate to other user interface views of the application that include specific health data organized by category. In some examples, each of the category-specific user interface views may include one or more updatable user interface elements 502 or other suitable user interface element that may be used to present health data and may be updated according to the techniques described herein. The search bar 510 may be used to search the health data on the user device, which may be stored in a data model in one or more databases. The data model may include a personalized health data graph, which is specific to a user 512 (e.g., “Allison” in this example).

The summary view 504 may also include summary area 514. The summary area 514 may include information about the user 512, including, for example, a profile picture 516 and a status identifier 518 corresponding to the health data presented by the updatable user interface elements 502. In this example, the status identifier 518 indicates that the health data is “current.” Other status identifiers 518 may include, “updating,” “initializing,” “syncing,” “disconnected,” and other similar identifiers, which may depend on whether the health data is available, whether the user device 106 is has a connection with a health data sources, and the like. In some examples, a user input 519, which is illustrated as a pull down, may be used to update the summary view 504. In some examples, such an input may be used a trigger to implement the process 200 and determine whether to use animations to present new health data. The summary area 514 may also include an option for showing all health data 520.

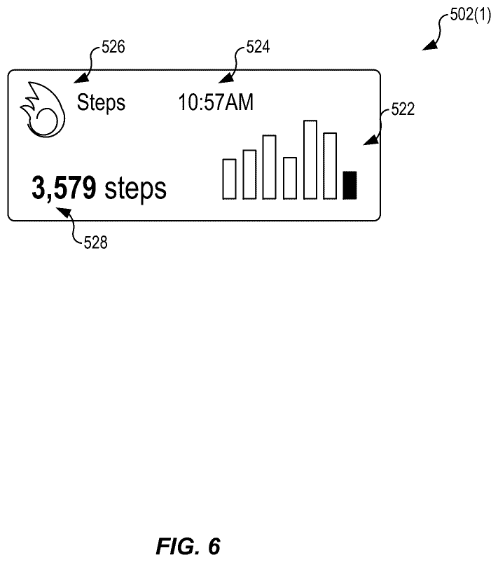

The updatable user interface elements 502 may be located at or near the top of the summary view 504 to prominently display these elements 502 in the view 504. In some examples, the updatable user interface elements 502 may be “pinned” or “favorited,” which may cause the user device 106 to generate the summary view 504 with these elements at their current locations. Examples of updatable user interface elements 502 may include a step-based updatable user interface element 502(1), a weight-based updatable user interface element 502(2), a heartrate-based updatable user interface element 502(3), and a steadiness-based updatable user interface element 502(4). Other updatable user interface element may be provided, which may represent other types or categories of health data. For example, workout goals, flights of stairs climbed, cholesterol, cycle tracking, wellness tracking, and the like may include their own updatable user interface elements 502. The details of an example updatable user interface element 502 will be described with respect to FIG. 6.

FIG. 6 illustrates a zoomed-in view of an example updatable user interface element 502(1) from FIG. 5, according to at least one example. While the updatable user interface element 502(1) is described in FIG. 5, the discussion is equally applicable to the other updatable user interface elements 502. The updatable user interface element 502(1) represents step data. In particular, the updatable user interface element 502(1) may include a graphical area 522 in which may be presented graphical information that relates to the health data. For example, the graphical area 522 may include a graph that represents the user's step data over some period of time (e.g., last seven days). The updatable user interface element 502(1) may also include a date field 524 to show when the data was last updated. The updatable user interface element 502(1) may also include a graphical indicator 526, which may correspond to the type of health data represented by the updatable user interface element 502(1). The updatable user interface element 502(1) may also include a data type area 528. In the updatable user interface element 502(1), the data type area 528 includes the double type data type used for step data.

Turning now to FIGS. 7A-7C, which illustrate various states of a set of updatable user interface elements from FIG. 5, according to various examples. In particular, FIG. 7A illustrates the updatable user interface elements 502(1)-502(4) in a first state 700, FIG. 7B illustrates the updatable user interface elements 502(1)-502(4) in a second state 702, and FIG. 7C illustrates the updatable user interface elements 502(1)-502(4) in a third state 704. The first state 700 may correspond to an old state, a pre-updated state, or an otherwise current state in which the health data is shown as the old data. The second state 702 may correspond to a transition state in which the health data (e.g., values, graphs, and dates) are being updated using an animation as a presentation mode. The third state 704 may correspond to a new state in which the health data is shown as the new data.

In the first state 700 and with particular emphasis on the updatable user interface element 502(1), the data type area 528 includes a value of “3,579 steps,” the graphical area 522 includes a current bar graph, and the date field 524 includes a time of “10:56 AM”. The other updatable user interface elements 502 also have current data.

In the second state 702, the data type area 528 of the updatable user interface element 502(1) includes a ticker-up type transition occurring in the step value number. This is indicated by the number in the hundreds place ticking up from 5 to 8. While not illustrated, the numbers in the tens and units place may also increment to reach the new value in the third state 704. The updatable user interface element 502(3) also illustrates a ticker-up transition. The updatable user interface element 502(2) illustrates a ticker-down transition. The graphical area 522 is also updated between states 700 and 704 to illustrate that the values have increased. Similarly, the graphical areas of the other the updatable user interface elements 502 may be updated.

The graphical animations described herein may be implemented using any suitable technique. For example, a declarative software development framework for building user interfaces may be used. Such a framework may include a declarative syntax, composable and reusable views, state management, and event integration. In some examples, the view layer may include and/or be programmed using the framework.

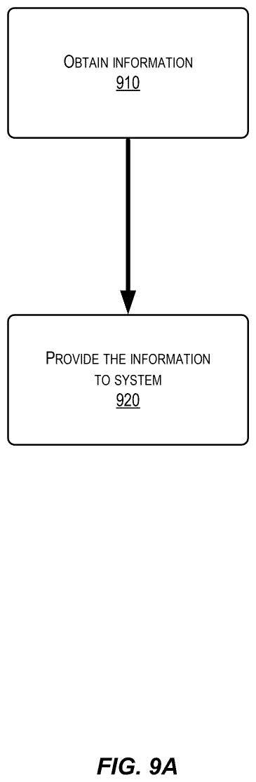

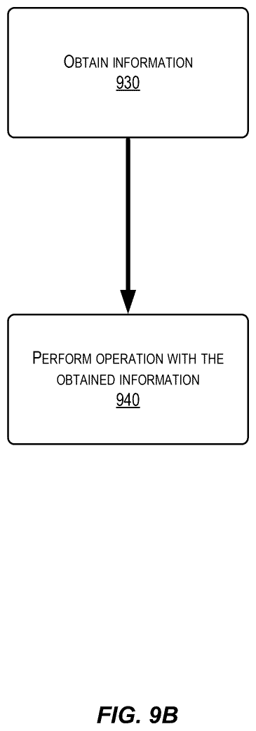

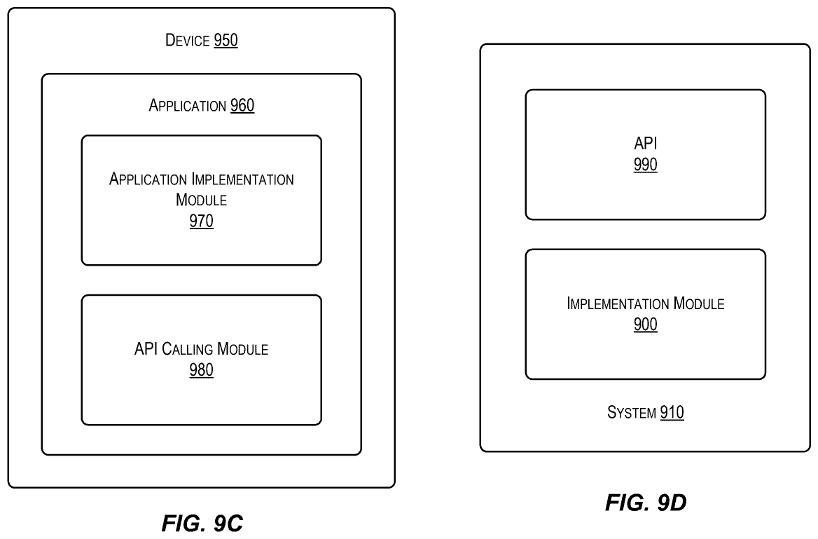

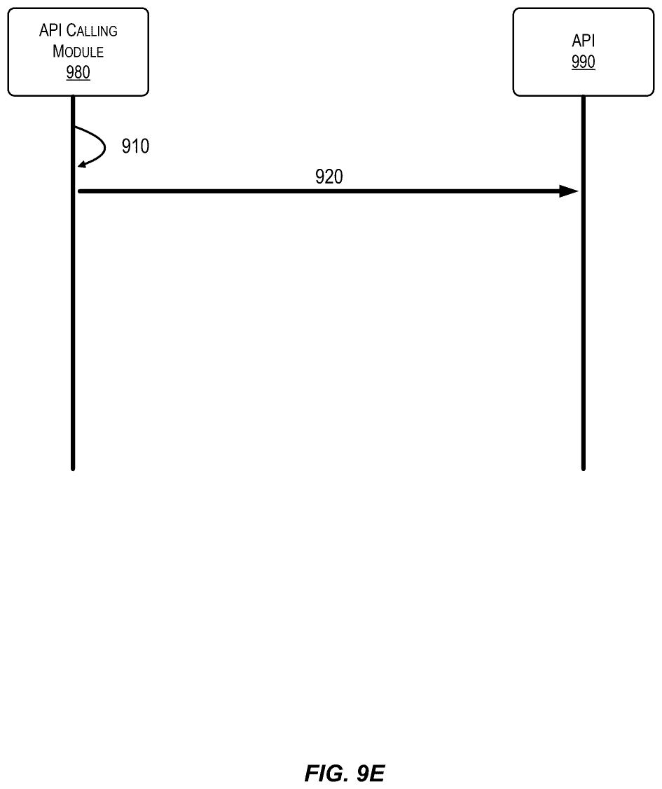

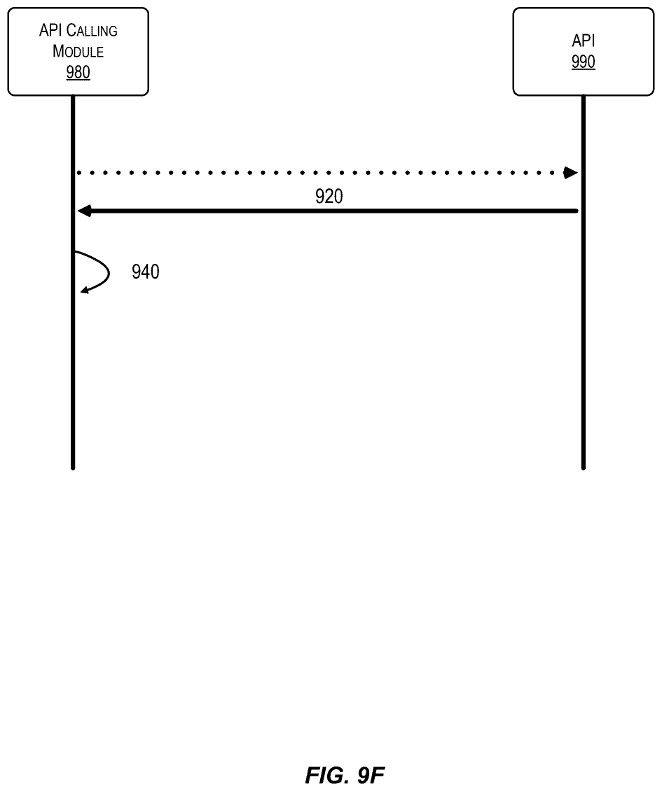

FIG. 8 illustrates a flow chart showing an example process 800 for selective determination of presentation modes for observation data elements, according to at least one example. The process 800 may be performed by the user device 106 (FIG. 1). In some examples, at least some portion (or the entirety thereof) may be performed by a service provider 902 (FIG. 9).

The process 800 begins at block 802 by the user device 106 receiving a new observation data element of a first data element data category. In some examples, block 802 may be performed at a data model layer of an application of the user device 106. The observation data element may correspond to health data. The first data element category may relate to a category of the health data, as described herein. In some examples, the first data element data category corresponds to a predefined standard for health data, which may include standards for organizing, storing, sharing, and retrieving health data. This can include, for example, use of a standardized health framework or other health standard (e.g., Health Level 7 (HL7), Logical Observation Identifiers Names and Codes (LOINC), Systematized Nomenclature of Medicine-Clinical Terms (SNOMED CT), International Classification of Disease (ICD), Current Procedural Terminology (CPT), RxNorm, and the like).

At block 804, the process 800 may include the user device 106 adding the new observation data element to a data model stored on the user device 106. The data model may include a personalized data graph that is stored on the user device 106. The personalized data graph may be used to store the health data of a user who is associated with the user device 106. In some examples, block 804 may be performed at the data model layer of the application of the user device 106.

At block 806, the process 800 may include the user device 106 generating bundled observation data. Generating the bundled observation data may be based on the new observation data element. In some examples, the bundled observation data may represent a new state for the new observation data element. In some examples, block 806 may be performed at the data model layer of the application of the user device 106.

In some examples, generating the bundled observation data at block 806 may include combining the new observation data element with an old observation data element of the first element data type to generate the bundled observation data. The old observation data element may represent an old state for the old observation data element.

At block 808, the process 800 may include the user device 106 providing the bundled observation data to a view layer of the application. In some examples, the block 808 is omitted from the process 800. In some examples, block 808 may be performed at the data model layer of the application of the user device 106.

At block 810, the process 800 may include the user device 106 evaluating the bundled observation data in accordance with at least one evaluation criteria. In some examples, the at least one evaluation criteria may be one of a plurality of evaluation criteria. The plurality of evaluation criteria may be used to determine a presentation mode relating to presentation of information associated with the bundled observation data. In some examples, block 810 may be performed at a view layer of the application of the user device 106.

At block 812, the process 800 may include the user device 106, implementing a presentation mode. In some examples, implementing the presentation mode may be in response to receiving a view input request. The view input request may include a user input request that includes a scroll input request received within a user interface view of the user interface of the application. In some examples, the view input request may include a user interface view request to view a user interface view of the user interface. The user interface view may include information corresponding to multiple observation data elements.

In some examples, the results of the evaluation at block 810 may be used to select the presentation mode. In some examples, the presentation mode may be implemented at the user interface of the user device. In some examples, block 812 may be performed at a view layer of the application of the user device 106.

In some examples, evaluating the bundled observation data in accordance with the at least one evaluation criteria to determine the presentation mode may include at least one of (i) determining that the old observation data element is null, or (ii) determining that the old observation data element is the same as the new observation data element. In some examples, this may correspond to block 218. In this example, implementing the presentation mode at the user interface of the user device may include updating the user interface to include information corresponding to the old observation data element without using a first animation effect.

In some examples, evaluating the bundled observation data in accordance with the at least one evaluation criteria to determine the presentation mode may include determining that the bundled observation data is of a first data type. In some examples, this may correspond to block 222. In this example, implementing the presentation mode at the user interface of the user device may include updating the user interface to include information corresponding to the new observation data element using a second animation effect. In some examples, the second animation effect may include a crossfade animation.

In some examples, evaluating the bundled observation data in accordance with the at least one evaluation criteria to determine the presentation mode may include determining that a first value of the new observation data element is less than a second value of the old observation data element. In some examples, this may correspond to block 226. In this example, implementing the presentation mode at the user interface of the user device may include updating the user interface to include information corresponding to the first value using a third animation effect. In some examples, the third animation effect may include a ticker-down animation.

In some examples, evaluating the bundled observation data in accordance with the at least one evaluation criteria to determine the presentation mode may include determining that a first value of the new observation data element is greater than a second value of the old observation data element. In some examples, this may correspond to block 226. In this example, implementing the presentation mode at the user interface of the user device may include updating the user interface to include information corresponding to the first value using a fourth animation effect. In some examples, the fourth animation effect may include a ticker-up animation.

In some examples, the application may include a health application, the user device may include a smartphone or a tablet, and the new observation data element may include user health-related data.

Implementations within the scope of the present disclosure can be partially or entirely realized using a tangible computer-readable storage medium (or multiple tangible computer-readable storage media of one or more types) encoding one or more computer-readable instructions. It should be recognized that computer-executable instructions can be organized in any format, including applications, widgets, processes, software, and/or components.

Implementations within the scope of the present disclosure include a computer-readable storage medium that encodes instructions organized as an application (e.g., application 960) that, when executed by one or more processing units, control an electronic device (e.g., device 950) to perform the method of FIG. 9A, the method of FIG. 9B, and/or one or more other processes and/or methods described herein.

It should be recognized that application 960 (shown in FIG. 9C) can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application. In some embodiments, application 960 is an application that is pre-installed on device 950 at purchase (e.g., a first party application). In other embodiments, application 960 is an application that is provided to device 950 via an operating system update file (e.g., a first party application or a second party application). In other embodiments, application 960 is an application that is provided via an application store. In some embodiments, the application store can be an application store that is pre-installed on device 950 at purchase (e.g., a first party application store). In other embodiments, the application store is a third-party application store (e.g., an application store that is provided by another application store, downloaded via a network, and/or read from a storage device).

Referring to FIG. 9A and FIG. 9E, application 960 obtains information (e.g., 910). In some embodiments, at 910, information is obtained from at least one hardware component of the device 950. In some embodiments, at 910, information is obtained from at least one software module of the device 950. In some embodiments, at 910, information is obtained from at least one hardware component external to the device 950 (e.g., a peripheral device, an accessory device, a server, etc.). In some embodiments, the information obtained at 910 includes positional information, health information, time information, notification information, user information, environment information, electronic device state information, weather information, media information, historical information, event information, hardware information, and/or motion information. In some embodiments, in response to and/or after obtaining the information at 910, application 960 provides the information to a system (e.g., 920). For example, the information at 910 may include a new observation data element of a first data element data.

In some examples, providing the information at 920 may include providing the first information to an operating system. The first information may be information for: i) adding the new observation data element to a data model stored on the user device; ii) generating bundled observation data based on the new observation data element, the bundled observation data representing a new state for the new observation data element; iii) providing the bundled observation data to a view layer of the application; iv) evaluating the bundled observation data in accordance with at least one evaluation criteria of a plurality of evaluation criteria to determine a presentation mode relating to presentation of information associated with the bundled observation data; and v) in response to receiving a view input request, implementing the presentation mode at a user interface of the user device.

In some embodiments, the system (e.g., 910 shown in FIG. 9D) is an operating system hosted on the device 950. In some embodiments, the system (e.g., 910 shown in FIG. 9D) is an external device (e.g., a server, a peripheral device, an accessory, a personal computing device, etc.) that includes an operating system.

Referring to FIG. 9B and FIG. 9F, application 960 obtains information (e.g., 930). In some embodiments, the information obtained at 930 includes positional information, time information, notification information, health information, user information, environment information electronic device state information, weather information, media information, historical information, event information, hardware information and/or motion information. In some examples, the information may include a new observation data element of a first data element data.

In response to and/or after obtaining the information at 930, application 960 performs an operation with the information (e.g., 940). In some embodiments, the operation performed at 940 includes: providing a notification based on the information, sending a message based on the information, displaying the information, controlling a user interface of a fitness application based on the information, controlling a user interface of a health application based on the information, controlling a focus mode based on the information, setting a reminder based on the information, adding a calendar entry based on the information, and/or calling an API of system 910 based on the information. In some examples, the information is based on: i) adding the new observation data element to a data model stored on the user device; ii) generating bundled observation data based on the new observation data element, the bundled observation data representing a new state for the new observation data element; iii) providing the bundled observation data to a view layer of the application; iv) evaluating the bundled observation data in accordance with at least one evaluation criteria of a plurality of evaluation criteria to determine a presentation mode relating to presentation of information associated with the bundled observation data; and v) in response to receiving a view input request, implementing the presentation mode at a user interface of the user device.

In some embodiments, one or more steps of the method of FIG. 9A and/or the method of FIG. 9B is performed in response to a trigger. In some embodiments, the trigger includes detection of an event, a notification received from system 910, a user input, and/or a response to a call to an API provided by system 910.

In some embodiments, the instructions of application 960, when executed, control device 950 to perform the method of FIG. 9A and/or the method of FIG. 9B by calling an application programming interface (API) (e.g., API 990) provided by system 910. In some embodiments, application 960 performs at least a portion of the method of FIG. 9A and/or the method of FIG. 9B without calling API 990.

In some embodiments, one or more steps of the method of FIG. 9A and/or the method of FIG. 9B includes calling an API (e.g., API 990) using one or more parameters defined by the API. In some embodiments, the one or more parameters include a constant, a key, a data structure, an object, an object class, a variable, a data type, a pointer, an array, a list or a pointer to a function or method, and/or another way to reference a data or other item to be passed via the API.

Referring to FIG. 9C, device 950 is illustrated. In some embodiments, device 950 is a personal computing device, a smart phone, a smart watch, a fitness tracker, a head mounted display (HMD) device, a media device, a communal device, a speaker, a television, and/or a tablet. As illustrated in FIG. 9C, device 950 includes application 960 and operating system (e.g., system 910 shown in FIG. 9D). Application 960 includes application implementation module 970 and API calling module 980. System 910 includes API 990 and implementation module 900. It should be recognized that device 950, application 960, and/or system 910 can include more, fewer, and/or different components than illustrated in FIGS. 9C and 9D.

In some embodiments, application implementation module 970 includes a set of one or more instructions corresponding to one or more operations performed by application 960. For example, when application 960 is a messaging application, application implementation module 970 can include operations to receive and send messages. In some embodiments, application implementation module 970 communicates with API calling module to communicate with system 910 via API 990 (shown in FIG. 9D).

In some embodiments, API 990 is a software module (e.g., a collection of computer-readable instructions) that provides an interface that allows a different module (e.g., API calling module 980) to access and/or use one or more functions, methods, procedures, data structures, classes, and/or other services provided by implementation module 900 of system 910. For example, API-calling module 980 can access a feature of implementation module 900 through one or more API calls or invocations (e.g., embodied by a function or a method call) exposed by API 990 and can pass data and/or control information using one or more parameters via the API calls or invocations. In some embodiments, API 990 allows application 960 to use a service provided by a Software Development Kit (SDK) library. In other embodiments, application 960 incorporates a call to a function or method provided by the SDK library and provided by API 990 or uses data types or objects defined in the SDK library and provided by API 990. In some embodiments, API-calling module 980 makes an API call via API 990 to access and use a feature of implementation module 900 that is specified by API 990. In such embodiments, implementation module 900 can return a value via API 990 to API-calling module 980 in response to the API call. The value can report to application 960 the capabilities or state of a hardware component of device 950, including those related to aspects such as input capabilities and state, output capabilities and state, processing capability, power state, storage capacity and state, and/or communications capability. In some embodiments, API 990 is implemented in part by firmware, microcode, or other low level logic that executes in part on the hardware component.

In some embodiments, API 990 allows a developer of API-calling module 980 (which can be a third-party developer) to leverage a feature provided by implementation module 900. In such embodiments, there can be one or more API-calling modules (e.g., including API-calling module 980) that communicate with implementation module 900. In some embodiments, API 990 allows multiple API-calling modules written in different programming languages to communicate with implementation module 900 (e.g., API 990 can include features for translating calls and returns between implementation module 900 and API-calling module 980) while API 990 is implemented in terms of a specific programming language. In some embodiments, API-calling module 980 calls APIs from different providers such as a set of APIs from an OS provider, another set of APIs from a plug-in provider, and/or another set of APIs from another provider (e.g., the provider of a software library) or creator of the another set of APIs.

Examples of API 990 can include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a bluetooth API, an NFC API, a UWB API, a fitness API, a smart home API, contact transfer API, photos API, camera API, and/or image processing API. In some embodiments the sensor API is an API for accessing data associated with a sensor of device 950. For example, the sensor API can provide access to raw sensor data. For another example, the sensor API can provide data derived (and/or generated) from the raw sensor data. In some embodiments, the sensor data includes temperature data, image data, video data, audio data, heart rate data, IMU (inertial measurement unit) data, lidar data, location data, GPS data, and/or camera data. In some embodiments, the sensor includes one or more of an accelerometer, temperature sensor, infrared sensor, optical sensor, heartrate sensor, barometer, gyroscope, proximity sensor, temperature sensor and/or biometric sensor.

In some embodiments, implementation module 900 is a system (e.g., operating system, server system) software module (e.g., a collection of computer-readable instructions) that is constructed to perform an operation in response to receiving an API call via API 990. In some embodiments, implementation module 900 is constructed to provide an API response (via API 990) as a result of processing an API call. By way of example, implementation module 900 and API-calling module 180 can each be any one of an operating system, a library, a device driver, an API, an application program, or other module. It should be understood that implementation module 900 and API-calling module 980 can be the same or different type of module from each other. In some embodiments, implementation module 900 is embodied at least in part in firmware, microcode, or other hardware logic.

In some embodiments, implementation module 900 returns a value through API 990 in response to an API call from API-calling module 980. While API 990 defines the syntax and result of an API call (e.g., how to invoke the API call and what the API call does), API 990 might not reveal how implementation module 900 accomplishes the function specified by the API call. Various API calls are transferred via the one or more application programming interfaces between API-calling module 980 and implementation module 900. Transferring the API calls can include issuing, initiating, invoking, calling, receiving, returning, and/or responding to the function calls or messages. In other words, transferring can describe actions by either of API-calling module 980 or implementation module 900. In some embodiments, a function call or other invocation of API 990 sends and/or receives one or more parameters through a parameter list or other structure.

In some embodiments, implementation module 900 provides more than one API, each providing a different view of or with different aspects of functionality implemented by implementation module 900. For example, one API of implementation module 900 can provide a first set of functions and can be exposed to third party developers, and another API of implementation module 900 can be hidden (e.g., not exposed) and provide a subset of the first set of functions and also provide another set of functions, such as testing or debugging functions which are not in the first set of functions. In some embodiments, implementation module 900 calls one or more other components via an underlying API and thus be both an API calling module and an implementation module. It should be recognized that implementation module 900 can include additional functions, methods, classes, data structures, and/or other features that are not specified through API 990 and are not available to API calling module 980. It should also be recognized that API calling module 980 can be on the same system as implementation module 900 or can be located remotely and access implementation module 900 using API 990 over a network. In some embodiments, implementation module 900, API 990, and/or API-calling module 980 is stored in a machine-readable medium, which includes any mechanism for storing information in a form readable by a machine (e.g., a computer or other data processing system). For example, a machine-readable medium can include magnetic disks, optical disks, random access memory, read only memory, and/or flash memory devices.

In some embodiments, process 800 (FIG. 8) is performed at a first computer system (as described herein) via a system process (e.g., an operating system process, a server system process) that is different from one or more applications executing and/or installed on the first computer system.

In some embodiments, method 800 (FIG. 8) is performed at a first computer system (as described herein) by an application that is different from a system process. In some embodiments, the instructions of the application, when executed, control the first computer system to perform method 800 (FIG. 8) by calling an application programming interface (API) provided by the system process. In some embodiments, the application performs at least a portion of method 800 without calling the API.

In some embodiments, the application can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application.

In some embodiments, the application is an application that is pre-installed on the first computer system at purchase (e.g., a first party application). In other embodiments, the application is an application that is provided to the first computer system via an operating system update file (e.g., a first party application). In other embodiments, the application is an application that is provided via an application store. In some implementations, the application store is pre-installed on the first computer system at purchase (e.g., a first party application store) and allows download of one or more applications. In some embodiments, the application store is a third party application store (e.g., an application store that is provided by another device, downloaded via a network, and/or read from a storage device). In some embodiments, the application is a third party application (e.g., an app that is provided by an application store, downloaded via a network, and/or read from a storage device). In some embodiments, the application controls the first computer system to perform method 800 (FIG. 8) by calling an application programming interface (API) provided by the system process using one or more parameters.

In some embodiments, exemplary APIs provided by the system process include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a bluetooth API, an NFC API, a UWB API, a fitness API, a smart home API, contact transfer API, photos API, camera API, and/or image processing API.

In some embodiments, at least one API is a software module (e.g., a collection of computer-readable instructions) that provides an interface that allows a different module (e.g., API calling module) to access and use one or more functions, methods, procedures, data structures, classes, and/or other services provided by an implementation module of the system process. The API can define one or more parameters that are passed between the API calling module and the implementation module. In some embodiments, the API 990 defines a first API call that can be provided by API calling module 990, wherein the definition for the first API call specifies the following call parameters: user id, get new health data, and any other suitable parameters. The implementation module is a system software module (e.g., a collection of computer-readable instructions) that is constructed to perform an operation in response to receiving an API call via the API. In some embodiments, the implementation module is constructed to provide an API response (via the API) as a result of processing an API call. In some embodiments, the implementation module is included in the device (e.g., 950) that runs the application. In some embodiments, the implementation module is included in an electronic device that is separate from the device that runs the application.

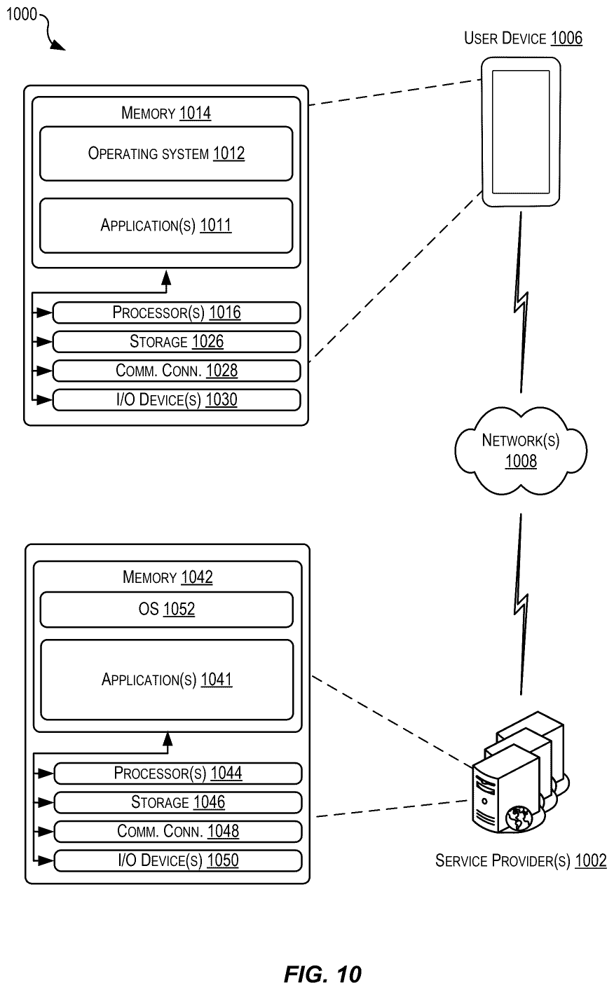

FIG. 10 illustrates an example architecture or environment 1000 configured to implement techniques described herein, according to at least one example. In some examples, the example architecture 1000 may further be configured to enable a user device 1006 and service provider computer 1002 to share information. The service provider computer 1002 is an example of a data source 104. The service provider computer 1002 may also provide other services and data for interacting with the user device 1006. The user device 1006 is an example of the user device 106. In some examples, the devices may be connected via one or more networks 1008 (e.g., via Bluetooth, WiFi, the Internet). In some examples, the service provider computer 1002 may be configured to implement at least some of the techniques described herein with reference to the user device 1006 and vice versa.

In some examples, the networks 1008 may include any one or a combination of many different types of networks, such as cable networks, the Internet, wireless networks, cellular networks, satellite networks, other private and/or public networks, or any combination thereof. While the illustrated example represents the user device 1006 accessing the service provider computer 1002 via the networks 1008, the described techniques may equally apply in instances where the user device 1006 interacts with the service provider computer 1002 over a landline phone, via a kiosk, or in any other manner. It is also noted that the described techniques may apply in other client/server arrangements (e.g., set-top boxes), as well as in non-client/server arrangements (e.g., locally stored applications, peer-to-peer configurations).

As noted above, the user device 1006 may be any type of computing device such as, but not limited to, a mobile phone, a smartphone, a personal digital assistant (PDA), a laptop computer, a desktop computer, a thin-client device, a tablet computer, a wearable device such as a smart watch, or the like. In some examples, the user device 1006 may be in communication with the service provider computer 1002 via the network 1008 or via other network connections.

In one illustrative configuration, the user device 1006 may include at least one memory 1014 and one or more processing units (or processor(s)) 1016. The processor(s) 1016 may be implemented as appropriate in hardware, computer-executable instructions, firmware, or combinations thereof. Computer-executable instruction or firmware implementations of the processor(s) 1016 may include computer-executable or machine-executable instructions written in any suitable programming language to perform the various functions described. The user device 1006 may also include geo-location devices (e.g., a global positioning system (GPS) device or the like) for providing and/or recording geographic location information associated with the user device 1006.

The memory 1014 may store program instructions that are loadable and executable on the processor(s) 1016, as well as data generated during the execution of these programs. Depending on the configuration and type of the user device 1006, the memory 1014 may be volatile (such as random access memory (RAM)) and/or non-volatile (such as read-only memory (ROM), flash memory). The user device 1006 may also include additional removable storage and/or non-removable storage 1026 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 1014 may include multiple different types of memory, such as static random access memory (SRAM), dynamic random access memory (DRAM), or ROM. While the volatile memory described herein may be referred to as RAM, any volatile memory that would not maintain data stored therein once unplugged from a host and/or power would be appropriate.

The memory 1014 and the additional storage 1026, both removable and non-removable, are all examples of non-transitory computer-readable storage media. For example, non-transitory computer-readable storage media may include volatile or non-volatile, removable or non-removable media implemented in any method or technology for storage of information such as computer-readable instructions, data structures, program modules, or other data. The memory 1014 and the additional storage 1026 are both examples of non-transitory computer-storage media. Additional types of computer-storage media that may be present in the user device 1006 may include, but are not limited to, phase-change RAM (PRAM), SRAM, DRAM, RAM, ROM, Electrically Erasable Programmable Read-Only Memory (EEPROM), flash memory or other memory technology, compact disc read-only memory (CD-ROM), digital video disc (DVD) or other optical storage, magnetic cassettes, magnetic tape, magnetic disk storage or other magnetic storage devices, or any other medium that can be used to store the desired information and that can be accessed by the user device 1006. Combinations of any of the above should also be included within the scope of non-transitory computer-readable storage media. Alternatively, computer-readable communication media may include computer-readable instructions, program modules, or other data transmitted within a data signal, such as a carrier wave, or other transmission. However, as used herein, computer-readable storage media does not include computer-readable communication media.

The user device 1006 may also contain communications connection(s) 1028 that allow the user device 1006 to communicate with a data store, another computing device or server, user terminals, and/or other devices via the network 1008. The user device 1006 may also include I/O device(s) 1030, such as a keyboard, a mouse, a pen, a voice input device, a touch screen input device, a display, speakers, and a printer.

Turning to the contents of the memory 1014 in more detail, the memory 1014 may include an operating system 1012 and/or one or more application programs or services for implementing the features disclosed herein such as applications 1011 (e.g., health application, digital wallet, third-party applications, browser application, and any other suitable application). In some examples, the applications 1011 may include a health application to perform similar techniques as described with reference to the user device 106. Similarly, at least some techniques described with reference to the service provider computer 1002 may be performed by the user device 106.

The service provider computer 1002 may also be any type of computing device such as, but not limited to, a collection of virtual or “cloud” computing resources, a remote server, a mobile phone, a smartphone, a PDA, a laptop computer, a desktop computer, a thin-client device, a tablet computer, a wearable device, a server computer, or a virtual machine instance. In some examples, the service provider computer 1002 may be in communication with the user device 1006 via the network 1008 or via other network connections.

In one illustrative configuration, the service provider computer 1002 may include at least one memory 1042 and one or more processing units (or processor(s)) 1044. The processor(s) 1044 may be implemented as appropriate in hardware, computer-executable instructions, firmware, or combinations thereof. Computer-executable instruction or firmware implementations of the processor(s) 1044 may include computer-executable or machine-executable instructions written in any suitable programming language to perform the various functions described.

The memory 1042 may store program instructions that are loadable and executable on the processor(s) 1044, as well as data generated during the execution of these programs. Depending on the configuration and type of service provider computer 1002, the memory 1042 may be volatile (such as RAM) and/or non-volatile (such as ROM and flash memory). The service provider computer 1002 may also include additional removable storage and/or non-removable storage 1046 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 1042 may include multiple different types of memory, such as SRAM, DRAM, or ROM. While the volatile memory described herein may be referred to as RAM, any volatile memory that would not maintain data stored therein, once unplugged from a host and/or power, would be appropriate. The memory 1042 and the additional storage 1046, both removable and non-removable, are both additional examples of non-transitory computer-readable storage media.

The service provider computer 1002 may also contain communications connection(s) 1048 that allow the service provider computer 1002 to communicate with a data store, another computing device or server, user terminals, and/or other devices via the network 1008. The service provider computer 1002 may also include I/O device(s) 1050, such as a keyboard, a mouse, a pen, a voice input device, a touch input device, a display, speakers, and a printer.

Turning to the contents of the memory 1042 in more detail, the memory 1042 may include an operating system 1052 and/or one or more application programs 1041 or services for implementing the features disclosed herein.

The various examples can be further implemented in a wide variety of operating environments, which in some cases can include one or more user computers, computing devices, or processing devices which can be used to operate any of a number of applications. User or client devices can include any of a number of general-purpose personal computers, such as desktop or laptop computers running a standard operating system, as well as cellular, wireless, and handheld devices running mobile software and capable of supporting a number of networking and messaging protocols. Such a system also can include a number of workstations running any of a variety of commercially available operating systems and other known applications for purposes such as development and database management. These devices also can include other electronic devices, such as dummy terminals, thin-clients, gaming systems, and other devices capable of communicating via a network.

Most examples utilize at least one network that would be familiar to those skilled in the art for supporting communications using any of a variety of commercially available protocols, such as TCP/IP, OSI, FTP, UPnP, NFS, CIFS, and AppleTalk. The network can be, for example, a local area network, a wide-area network, a virtual private network, the Internet, an intranet, an extranet, a public switched telephone network, an infrared network, a wireless network, and any combination thereof.

In examples utilizing a network server, the network server can run any of a variety of server or mid-tier applications, including HTTP servers, FTP servers, CGI servers, data servers, Java servers, and business application servers. The server(s) may also be capable of executing programs or scripts in response to requests from user devices, such as by executing one or more applications that may be implemented as one or more scripts or programs written in any programming language, such as Java®, C, C# or C++, or any scripting language, such as Perl, Python, or TCL, as well as combinations thereof. The server(s) may also include database servers, including without limitation those commercially available from Oracle®, Microsoft®, Sybase®, and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of examples, the information may reside in a storage-area network (SAN) familiar to those skilled in the art. Similarly, any necessary files for performing the functions attributed to the computers, servers, or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch screen, keypad), and at least one output device (e.g., a display device, printer, speaker). Such a system may also include one or more storage devices, such as disk drives, optical storage devices, and solid-state storage devices such as RAM or ROM, as well as removable media devices, memory cards, or flash cards.

Such devices can also include a computer-readable storage media reader, a communications device (e.g., a modem, a network card (wireless or wired), an infrared communication device), and working memory as described above. The computer-readable storage media reader can be connected with, or configured to receive, a non-transitory computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or browser. It should be appreciated that alternate examples may have numerous variations from that described above. For example, customized hardware might also be used and/or particular elements might be implemented in hardware, software (including portable software, such as applets), or both. Further, connection to other computing devices such as network input/output devices may be employed.

Non-transitory storage media and computer-readable media for containing code, or portions of code, can include any appropriate media known or used in the art, including storage media, such as, but not limited to, volatile and non-volatile, removable and non-removable media implemented in any method or technology for storage of information such as computer-readable instructions, data structures, program modules, or other data, including RAM, ROM, EEPROM, flash memory or other memory technology, CD-ROM, DVD or other optical storage, magnetic cassettes, magnetic tape, magnetic disk storage or other magnetic storage devices, or any other medium which can be used to store the desired information and which can be accessed by a system device. Based at least in part on the disclosure and teachings provided herein, a person of ordinary skill in the art will appreciate other ways and/or methods to implement the various examples.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated examples thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “having,” “including,” and “containing” are to be construed as open-ended terms (e.g., meaning “including, but not limited to”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate examples of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Preferred examples of this disclosure are described herein, including the best mode known to the inventors for carrying out the disclosure. Variations of those preferred examples may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

As described above, one aspect of the present technology is receiving and presenting health data using unique presentation modes, depending on state of the data. The present disclosure contemplates that in some instances, this health data may include personally identifiable information (PII) data that uniquely identifies or can be used to contact or locate a specific person. Such personal information data can include demographic data, location-based data, telephone numbers, email addresses, Twitter IDs, home addresses, data or records relating to a user's health or level of fitness (e.g., vital sign measurements, medication information, exercise information), date of birth, health record data, or any other identifying or personal or health information.

The present disclosure recognizes that the use of such personal information data, in the present technology, can be used to the benefit of users. For example, the personal information data can be used to provide enhancements to a user's personal health record, including viewing health data. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the U.S., collection of or access to certain health data may be governed by federal and/or state laws, such as the Health Insurance Portability and Accountability Act (HIPAA); whereas health data in other countries may be subject to other regulations and policies and should be handled accordingly. Hence, different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services or other services relating to health record management, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health-related applications, data de-identification can be used to protect a user's privacy. De-identification may be facilitated, when appropriate, by removing specific identifiers (e.g., date of birth), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data.

What is claimed is:

1. A computer-implemented method, comprising:
at a data model layer of an application of a user device,
receiving a new observation data element of a first data element data category;
adding the new observation data element to a data model stored on the user device;
generating bundled observation data based on the new observation data element, the bundled observation data representing a new state for the new observation data element; and
providing the bundled observation data to a view layer of the application;
at the view layer of the application,
evaluating the bundled observation data in accordance with at least one evaluation criteria of a plurality of evaluation criteria to determine a presentation mode relating to presentation of information associated with the bundled observation data; and
in response to receiving a view input request, implementing the presentation mode at a user interface of the user device.

2. The computer-implemented method of claim 1, wherein generating the bundled observation data comprises combining the new observation data element with an old observation data element of the first data element data category to generate the bundled observation data, the old observation data element representing an old state for the old observation data element.

3. The computer-implemented method of claim 2, wherein evaluating the bundled observation data in accordance with the at least one evaluation criteria to determine the presentation mode comprises at least one of:
determining that the old observation data element is null; or
determining that the old observation data element is the same as the new observation data element, and wherein implementing the presentation mode at the user interface of the user device comprises updating the user interface to include information corresponding to the old observation data element without using a first animation effect.

4. The computer-implemented method of claim 2, wherein evaluating the bundled observation data in accordance with the at least one evaluation criteria to determine the presentation mode comprises determining that the bundled observation data is of a first data type, and wherein implementing the presentation mode at the user interface of the user device comprises updating the user interface to include information corresponding to the new observation data element using a second animation effect.

5. The computer-implemented method of claim 4, wherein the second animation effect comprises a crossfade animation.

6. The computer-implemented method of claim 2, wherein evaluating the bundled observation data in accordance with the at least one evaluation criteria to determine the presentation mode comprises determining that a first value of the new observation data element is less than a second value of the old observation data element, and wherein implementing the presentation mode at the user interface of the user device comprises updating the user interface to include information corresponding to the first value using a third animation effect.

7. The computer-implemented method of claim 6, wherein the third animation effect comprises a ticker-down animation.

8. The computer-implemented method of claim 2, wherein evaluating the bundled observation data in accordance with the at least one evaluation criteria to determine the presentation mode comprises determining that a first value of the new observation data element is greater than a second value of the old observation data element, and wherein implementing the presentation mode at the user interface of the user device comprises updating the user interface to include information corresponding to the first value using a fourth animation effect.

9. The computer-implemented method of claim 8, wherein the fourth animation effect comprises a ticker-up animation.

10. A user device, comprising:
a memory configured to store computer-executable instructions; and
a processor configured to access the memory and execute the computer-executable instructions to at least:
receive a new observation data element of a first data element data category;
add the new observation data element to a data model stored on the user device;
generate bundled observation data based on the new observation data element, the bundled observation data representing a new state for the new observation data element;
provide the bundled observation data to a view layer of the application;
evaluate the bundled observation data in accordance with at least one evaluation criteria of a plurality of evaluation criteria to determine a presentation mode relating to presentation of information associated with the bundled observation data; and
in response to receiving a view input request, implement the presentation mode at a user interface of the user device.

11. The user device of claim 10, wherein generating the bundled observation data comprises combining the new observation data element with an old observation data element of the first data element data category to generate the bundled observation data, the old observation data element representing an old state for the old observation data element.

12. The user device of claim 10, wherein the view input request comprises a scroll input request received within a user interface view of the user interface of the application.

13. The user device of claim 10, wherein the view input request comprises a user interface view request to view a user interface view of the user interface, the user interface view comprising information corresponding to multiple observation data elements.

14. The user device of claim 10, wherein:
the application comprises a health application;
the user device comprises a smartphone or a tablet; and
the new observation data element comprises user health-related data.

15. A non-transitory computer-readable storage medium comprising computer-executable instructions that, when executed by one or more processors of a user device, cause the user device to perform operations comprising:
receiving a new observation data element of a first data element data category;
adding the new observation data element to a data model stored on the user device;
generating bundled observation data based on the new observation data element, the bundled observation data representing a new state for the new observation data element;
providing the bundled observation data to a view layer of the application;
evaluating the bundled observation data in accordance with at least one evaluation criteria of a plurality of evaluation criteria to determine a presentation mode relating to presentation of information associated with the bundled observation data; and
in response to receiving a view input request, implementing the presentation mode at a user interface of the user device.

16. The non-transitory computer-readable storage medium of claim 15, wherein generating the bundled observation data comprises combining the new observation data element with an old observation data element of the first data element data category to generate the bundled observation data, the old observation data element representing an old state for the old observation data element.

17. The non-transitory computer-readable storage medium of claim 16, wherein evaluating the bundled observation data in accordance with the at least one evaluation criteria to determine the presentation mode comprises at least one of:
determining that the old observation data element is null; or
determining that the old observation data element is the same as the new observation data element, and wherein implementing the presentation mode at the user interface of the user device comprises updating the user interface to include information corresponding to the old observation data element without using a first animation effect.

18. The non-transitory computer-readable storage medium of claim 16, wherein evaluating the bundled observation data in accordance with the at least one evaluation criteria to determine the presentation mode comprises determining that the bundled observation data is of a first data type, and wherein implementing the presentation mode at the user interface of the user device comprises updating the user interface to include information corresponding to the new observation data element using a second animation effect, and wherein the second animation effect comprises a crossfade animation.

19. The non-transitory computer-readable storage medium of claim 16, wherein evaluating the bundled observation data in accordance with the at least one evaluation criteria to determine the presentation mode comprises determining that a first value of the new observation data element is less than a second value of the old observation data element, and wherein implementing the presentation mode at the user interface of the user device comprises updating the user interface to include information corresponding to the first value using a third animation effect, and wherein the third animation effect comprises a ticker-down animation.

20. The non-transitory computer-readable storage medium of claim 16, wherein evaluating the bundled observation data in accordance with the at least one evaluation criteria to determine the presentation mode comprises determining that a first value of the new observation data element is greater than a second value of the old observation data element, and wherein implementing the presentation mode at the user interface of the user device comprises updating the user interface to include information corresponding to the first value using a fourth animation effect, and wherein the fourth animation effect comprises a ticker-up animation.
